(19) United States
(12) Patent Application Publication
(10) Pub. No.: US 2021/0398622 A1
(43) Pub. Date: Dec. 23, 2021

(54) METHOD AND SYSTEM FOR ASSESSING DRUG USE IN CHILDREN, AND COMPUTING DEVICE

(71) Applicant: West China Second Univ. Hospital, Sichuan Univ., Chengdu (CN)

(72) Inventors: Lingli Zhang, Chengdu (CN); Linan Zeng, Chengdu (CN); Liang Huang, Chengdu (CN); Chunsong Yang, Chengdu (CN); Chuan Zhang, Chengdu (CN); Zhe Chen, Chengdu (CN); Dan Liu, Chengdu (CN); Hailong Li, Chengdu (CN); Zhenyan Bo, Chengdu (CN); Wenrui Li, Chengdu (CN); Mao Lin, Chengdu (CN); Fang Qin, Chengdu (CN)

(21) Appl. No.: 17/211,241

(22) Filed: Mar. 24, 2021

(30) Foreign Application Priority Data
Jun. 18, 2020 (CN) 202010561787.4

(51) Int. Cl. G16H 10/20; G16H 20/10

(52) U.S. Cl. CPC G16H 10/20; G16H 20/10

(57) ABSTRACT

The present disclosure relates to the technical field of data processing, and in particular to a method and a system for assessing drug use in children, and a computing device. The method includes: building a rational drug use (RDU) assessment model for clinical drug use in children; acquiring clinical drug use data of children and extracting classification information therefrom;

according to the classification information, importing the clinical drug use data of children correspondingly into the RDU assessment model for data comparison to obtain comparison results; and summarizing all comparison results according to the classification information to generate rationality assessment results for clinical drug use in children. The present disclosure can effectively improve the efficiency of rationality assessment for clinical drug use in children, so as to realize the monitoring of clinical drug use in children.

### TECHNICAL FIELD

The present disclosure relates to the technical field of data processing, and in particular to a method and a system for assessing drug use in children, and a computing device.

### BACKGROUND

The assessment of rational drug use (RDU) refers to the assessment of safety, effectiveness, economy, and appropriateness of clinical drug use.

Safety is not an absolute concept of whether a drug has toxic and side effects or whether a drug leads to adverse reactions; instead, it emphasizes that a user can acquire the greatest therapeutic effect with the least risk from medication.

Effectiveness refers to achieving an expected purpose through the action of a drug. Clinical effectiveness indicators include cure rate, marked improvement rate, improvement rate, inefficiency, and so on. Economy takes into account the cost and benefit of drug use, that is, the minimum drug expenditure is used to achieve the maximum therapeutic gain, and limited medical resources are rationally used to reduce a medical burden on a patient.

Appropriateness is an important indicator to achieve RDU, which includes appropriate indications, appropriate drugs, appropriate drug use (administration time, administration dosage, course of treatment, and administration route), appropriate patients, appropriate information, and appropriate observations.

It is particularly important to monitor and assess RDU in special patients, especially in child patients. However, the current RDU assessment and monitoring for clinical drug use of child patients mainly relies on manual assessment by senior experts. There has not yet been a unified assessment system to efficiently and automatically complete the RDU assessment for clinical drug use of child patients to realize the supervision of drug use for children.

### SUMMARY

In view of the deficiencies in the prior art, the present disclosure provides a method and a system for assessing drug use in children, and a computing device. The present disclosure, when in use, can effectively improve the efficiency of rationality assessment for clinical drug use in children, so as to realize the monitoring of clinical drug use in children.

In a first aspect, the present disclosure provides a method for assessing drug use in children, including:

building an RDU assessment model for clinical drug use in children; acquiring clinical drug use data of children and extracting classification information therefrom;

according to the classification information, importing the clinical drug use data of children correspondingly into the RDU assessment model for data comparison to obtain comparison results; and

summarizing all comparison results according to the classification information to generate rationality assessment results for clinical drug use in children.

Based on the above content of the present disclosure, an RDU assessment model for clinical drug use in children can be built, clinical drug use data of children can be imported into the RDU assessment model to obtain corresponding comparison results, and then the comparison results can be summarized to obtain rationality assessment results for clinical drug use in children. The method for assessing drug use in children can be used instead of the traditional manual assessment method to effectively improve the efficiency of rationality assessment for clinical drug use in children, so as to realize the supervision of clinical drug use in children.

In a possible design, a process of building an RDU assessment model for clinical drug use in children may include:

acquiring existing indicator data for drug use in children;

extracting key information from the existing indicator data for drug use in children, where, the key information may include child age group information, disease burden information, drug metabolism information, and drug use characteristic information; and

establishing an assessment indicator system for RDU in children according to the key formation, and building the RDU assessment model based on the assessment indicator system for RDU in children.

Based on the above content of the present disclosure, existing indicator data for drug use in children can be acquired to establish an assessment indicator system for RDU in children, and then an RDU assessment model is established based on the assessment indicator system for RDU in children, so that the RDU assessment model can have a wider assessment range.

In a possible design, the method may further include: using the Delphi method to obtain corrected data; and using the corrected data to correct and optimize the assessment indicator system for RDU in children.

Based on the above content of the present disclosure, the Delphi method can be used to consult senior experts in terms of the established assessment indicator system for RDU in children to obtain corresponding corrected data through statistical induction, and then the corrected data can be used to correct and optimize the assessment indicator system for RDU in children so that an assessment result obtained from the RDU assessment model is more accurate.

In a possible design, a process of acquiring clinical drug use data of children may include:

acquiring a prescription for clinical drug use in children;

reading a content in the prescription for drug use in children to obtain basic information about clinical drug use in children; and

sorting the basic information about clinical drug use in children to obtain the clinical drug use data of children.

Based on the above content of the present disclosure, a prescription for clinical drug use in children can be processed to obtain clinical drug use data of children, which is very convenient.

In a possible design, the method may further include:

determining rationality assessment results for clinical drug use in children in various hospitals;

according to the rationality assessment results for clinical drug use in children in various hospitals, generating corresponding rationality assessment reports for clinical drug use in children; and

feeding the corresponding rationality assessment reports for clinical drug use in children back to the hospitals, respectively.

Based on the above content of the present disclosure, after rationality assessment results for clinical drug use in children in various hospitals are obtained, corresponding results can be organized into rationality assessment reports for clinical drug use in children, which can be fed back to the hospitals, so as to realize the supervision of clinical drug use in children in the hospitals.

In a possible design, the classification information extracted from the clinical drug use data of children may include drug selection information, drug use course information, usage and dosage information, and drug cost information.

Based on the above content of the present disclosure, the rationality assessment of clinical drug use in children can be completed from multiple aspects, so that assessment results are more detailed and comprehensive.

In a second aspect, the present disclosure provides a system for assessing drug use in children, including:

a building unit configured to build an RDU assessment model for clinical drug use in children;

an acquisition unit configured to acquire clinical drug use data of children and extract classification information therefrom;

a comparison unit configured to import the clinical drug use data of children correspondingly into the RDU assessment model for data comparison according to the classification information to obtain comparison results; and

a summarization unit configured to summarize all comparison results according to the classification information to generate rationality assessment results for clinical drug use in children.

In a possible design, the acquisition unit may be also configured to acquire existing indicator data for drug use in children; and the building unit may be also configured to build an RDU assessment model for clinical drug use in children, and specifically configured to:

extract key information from the existing indicator data for drug use in children, where, the key information may include child age group information, disease burden information, drug metabolism information, and drug use characteristic information; and

establish an assessment indicator system for RDU in children according to the key information, and build the RDU assessment model based on the assessment indicator system for RDU in children.

In a possible design, the system may further include:

an acquisition unit configured to use the Delphi method to obtain corrected data; and

a correction unit configured to use the corrected data to correct and optimize the assessment indicator system for RDU in children.

In a possible design, the system may further include:

a determination unit configured to determine rationality assessment results for clinical drug use in children in various hospitals;

a generation unit configured to generate corresponding rationality assessment reports for clinical drug use in children according to the rationality assessment results for clinical drug use in children in various hospitals; and

a feedback unit configured to feed the corresponding rationality assessment reports for clinical drug use in children back to the hospitals, respectively.

In a third aspect, the present disclosure provides a computing device, including:

a memory configured to store instructions; and

a processor configured to read the instructions stored in the memory and execute any method described in the first aspect based on the instructions.

In a fourth aspect, the present disclosure provides a computer-readable storage medium. Instructions are stored on the computer-readable storage medium, and when the instructions are run on a computer, the computer executes any method described in the first aspect.

In a fifth aspect, the present disclosure provides a computer program product with instructions. When the instructions are run on a computer, the computer executes any method described in the first aspect.

### BRIEF DESCRIPTION OF DRAWINGS

To describe the technical solutions in examples of the present disclosure or in the prior art more clearly, the accompanying drawings required for describing the examples or the prior art will be briefly described below. Apparently, the accompanying drawings in the following description only show some examples of the present disclosure, and a person of ordinary skill in the art may still derive other drawings from these accompanying drawings without creative efforts.

FIG. 1 is a schematic flowchart for the method according to the present disclosure;

FIG. 2 is a schematic structural diagram for the system according to the present disclosure; and

FIG. 3 is a schematic structural diagram for the computing device according to the present disclosure.

### DETAILED DESCRIPTION

The present disclosure will be further described below with reference to the accompanying drawings and specific examples. It should be noted here that the description of these examples is used to help understand the present disclosure, but does not constitute a limitation to the present disclosure. The specific structural and functional details disclosed herein are only used to describe illustrative examples of the present disclosure. However, the present disclosure may be embodied in many alternative forms, and it should not be understood that the present disclosure is limited to the examples set forth herein.

It should be understood that the terms such as “first” and “second” are used only for distinguishing description and cannot be understood as indicating or implying relative importance. Although the terms such as “first” and “second” can be used herein to describe various units, these units should not be limited by these terms. These terms are only used to distinguish one unit from another. For example, a first unit may be referred to as a second unit, and a second unit may be referred to as a first unit similarly, without departing from the scope of the illustrative examples of the present disclosure.

It should be understood that the term “and/or” used herein only refers to an association relationship for describing associated objects and represents that there may be three relationships. For example, A and/or B may represent the following three cases: there is A alone; there is B alone; and there are both A and B. The term “/and” used herein refers to another relationship between associated objects and represents that there may be two relationships. For example, A/and B may represent the following two cases: there is A alone, and there is both A and B. In addition, the character “/” used herein generally indicates an “or” relationship between the associated objects before and after the character.

It should be understood that, in the description of the present disclosure, orientation or position relationships indicated by terms “upper”, “vertical”, “inner”, “outer”, etc. are usual orientation or position relationships of the products of the present disclosure when in use, or orientation or position relationships commonly understood by those skilled in the art. These terms are only used to facilitate description of the present disclosure and simplify the description, but not to indicate or imply that the mentioned device or components must have a specific orientation or must be established and operated in a specific orientation, and thus these terms cannot be understood as a limitation to the present disclosure.

It should be understood that, in the description where a unit is “connected with”, “connected to”, or “coupled with” another unit, the unit can be connected or coupled to the other unit directly or through an intermediate unit. In contrast, in the description where a unit is “directly connected” or “directly coupled” with another unit, there is no intermediate unit. Other words used to describe relationships among units should be interpreted in a similar way (for example, “between” vs “directly between”, “adjacent” vs “directly adjacent”, etc.).

In the description of the present disclosure, it should be noted that, unless otherwise clearly specified and limited, meanings of terms “disposed”, “installed”, and “connected with” should be understood in a board sense. For example, the connection may be a fixed connection, a removable connection, or an integral connection; may be a mechanical connection or an electrical connection; may be a direct connection or an indirect connection through an intermediate medium; or may be intercommunication between two components. A person of ordinary skill in the art may understand specific meanings of the foregoing terms in the present disclosure based on a specific situation.

The terms used herein are only used to describe specific examples, and are not intended to limit illustrative examples of the present disclosure. As used herein, the singular forms “a”, “an”, and “the” are intended to include plural forms, unless the context clearly indicates the opposite. It should also be understood that the terms “include”, “includes”, “comprises”, and/or “comprises”, when used herein, specify the existence of the stated features, integers, steps, operations, units, and/or components, which do not exclude the existence or addition of one or more other features, quantities, steps, operations, units, and components, and/or combinations thereof.

It should also be noted that, in some alternative examples, the functions/actions may appear in an order different from that shown in the figures. For example, some involved functions/actions may actually be executed substantially concurrently, or sometimes two figures shown in succession may be executed in a reverse order.

Specific details are provided in the following description to facilitate a complete understanding of the illustrative examples. However, those of ordinary skill in the art should understand that the illustrative examples can be implemented without these specific details. For example, the system can be shown in a block diagram to avoid the problem that an example is illustrated unclearly due to unnecessary details. In other examples, well-known procedures, structures, and technologies may not be shown in unnecessary details to avoid making the illustrative examples unclear.

### EXAMPLE 1

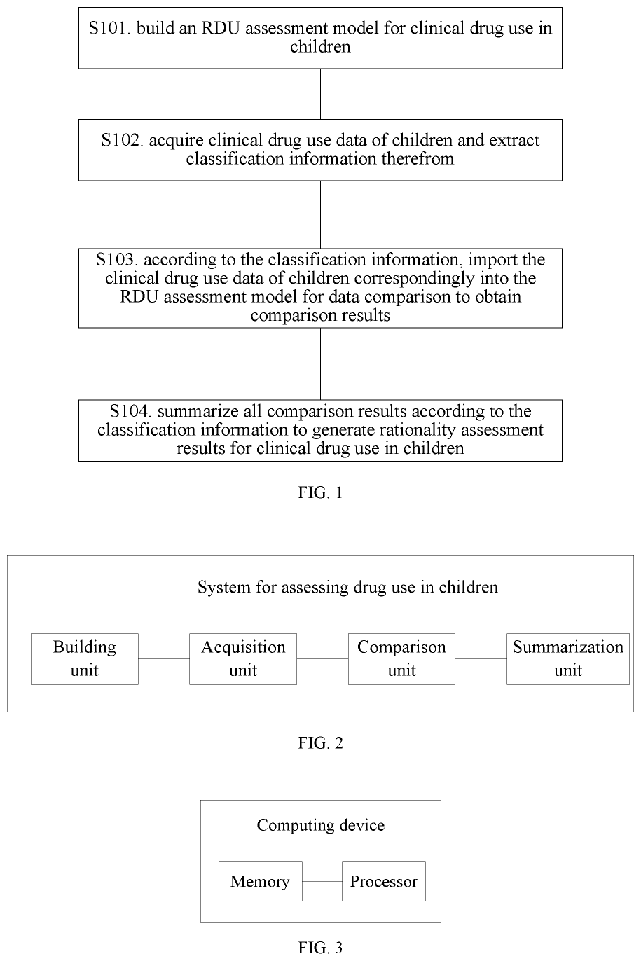

This example provides a method for assessing drug use in children, as shown in FIG. 1, including the following steps:

S101. An RDU assessment model is built for clinical drug use in children. A process of building an RDU assessment model for clinical drug use in children may include, but is not limited to:

acquiring existing indicator data for drug use in children worldwide through various channels; extracting key information from the existing indicator data for drug use in children, where, the key information may include child age group information, disease burden information, drug metabolism information, and drug use characteristic information; and establishing an assessment indicator system for RDU in children according to the key information, and building the RDU assessment model based on the assessment indicator system for RDU in children.

Moreover, the assessment indicator system for RDU in children can also be established with reference to an existing assessment system for RDU in adults.

The Delphi method can be used to obtain corresponding corrected data for the established assessment indicator system for RDU in children, and then the corrected data can be used to correct and optimize the assessment indicator system for RDU in children. The Delphi method mainly includes: anonymously soliciting opinions from experts through several rounds of letter inquiries and then drawing up a comprehensive result through a summary analysis. Generally speaking, the Delphi method is to make decisions or form an assessment system by summarizing opinions from experts.

S102. Clinical drug use data of children were acquired, and classification information is extracted therefrom.

A process of acquiring clinical drug use data of children may include, but is not limited to:

acquiring a prescription for clinical drug use in children;

reading a content in the prescription for drug use in children to obtain basic information about clinical drug use in children, including registration number, age, gender, drug selection, usage and dosage, administration route, administration frequency, drug use course, etiological examination result, drug cost, etc. of a child patient; and

sorting the basic information about clinical drug use in children to obtain the clinical drug use data of children.

S103. According to the classification information, the clinical drug use data of children are imported correspondingly into the RDU assessment model for data comparison to obtain comparison results.

The classification information may include drug selection information, drug use course information, usage and dosage information, and drug cost information, and the multi-aspect data comparison for clinical drug use data of children is achieved through classification information in multiple aspects. S104. All comparison results are summarized according to the classification information to generate rationality assessment results for clinical drug use in children.

In order to facilitate the supervision of clinical drug use in children in various hospitals, rationality assessment results for clinical drug use in children in various hospitals can be determined; then corresponding rationality assessment reports for clinical drug use in children are generated according to the rationality assessment results for clinical drug use in children in various hospitals; and the corresponding rationality assessment reports for clinical drug use in children are fed back to the hospitals, respectively.

### EXAMPLE 2

As an optimization to the above-mentioned example, an assessment indicator system for RDU in children is established based on common acute diseases, chronic diseases, and drug use categories (drug selection, usage and dosage, drug use course) in children. Children primary nephrotic syndrome (PNS), children community-acquired pneumonia (CAP), and children proton-pump inhibitor (PPI) are mainly selected for the rationality assessment of drug use. As an example, RDU assessment indicators for children with PNS are shown in Table 1 below:

TABLE 1

| | Outpatient | Inpatient |
|---|---|---|
| Drug | Use ratio of antibacterial drugs | Use ratio of antibacterial drugs |
| selection | Use ratio of renin-angiotensin system (RAS) inhibitors | Use ratio of RAS inhibitors among non-hypertensive |
| | among non-hypertensive child patients | child patients |
| | Use ratio of albumin | Use ratio of albumin |
| | Use ratio of immunopotentiators | Use ratio of immunopotentiators |
| | Use ratio of calcineurin inhibitors (CNIs) | Use ratio of CNIs |
| | Use ratio of calcium supplement treatment | Use ratio of calcium supplement treatment |
| | Use ratio of preferred prednisone (prednisolone) among child | Use ratio of preferred prednisone (prednisolone) |
| | patients undergoing hormone treatment | among child patients undergoing hormone treatment |
| | Use ratio of pulse hormone therapy | Use ratio of pulse hormone therapy |
| | Use ratio of CTX among child patients with | Use ratio of CTX among child patients with |
| | hormone-sensitive and frequent-relapse PNS that are | hormone-sensitive and frequent-relapse PNS that are |
| | administered with immunosuppressants | administered with immunosuppressants |
| | Use ratio of anticoagulation preventive measures (defined | Use ratio of anticoagulation preventive measures |
| | measures: warfarin, dipyridamole, Clopidogrel, aspirin, and | (defined measures: warfarin, dipyridamole, |
| | low-molecular-weight heparin (LMWH)) | Clopidogrel, aspirin, and LMWH) |
| Usage and | Initial hormone treatment: >60 mg/d | Initial hormone treatment: >60 mg/d |
| dosage | Use ratio of intravenous administration among child patients | Use ratio of intravenous administration among child |
| | taking diuretics | patients taking diuretics |
| | Use ratio of cyclophosphamide (CP) intravenous injection | Use ratio of CP intravenous injection among child |
| | among child patients taking CP | patients taking CP |
| | Use ratio of a combination of an angiotensin-converting | Use ratio of a combination of an ACEI/ARB with a |
| | enzyme inhibitor (ACEI)/angiotensin receptor blocker | diuretic |
| | (ARB) with a diuretic | |
| | | During initial treatment (three days before |
| | | admission), use ratio of diuretics |
| | Use ratio of blood concentration monitoring among patients | Use ratio of blood concentration monitoring among |
| | taking CNI drugs | patients taking CNI drugs |

RDU assessment indicators for children with CAP are shown in Table 2 below:

TABLE 2

| | Outpatient | Inpatient |
|---|---|---|
| Drug | Use ratio of antibiotics | Use ratio of antibiotics |
| selection | Use ratio of broad-spectrum antibiotics after | Use ratio of broad-spectrum antibiotics after pathogenic bacteria |
| | pathogenic bacteria are identified | are identified |
| | Use ratio of preferred β-lactam antibiotics | Use ratio of preferred β-lactam antibiotics |
| | Use ratio of macrolide antibiotics | Use ratio of macrolide antibiotics |
| | Use ratio of combined antibiotics | Use ratio of combined antibiotics |
| | Proportion of a combination of macrolides and | Proportion of a combination of macrolides and β-lactams in drug |
| | β-lactams in drug combination | combination |
| | | Use ratio of third-generation cephalosporin antibiotics |
| | | Use ratio of vancomycin or clindamycin when pathogenic |
| | | bacteria are identified as Staphylococcus aureus (S. aureus) |
| | | Microbiological examination rate among CAP child patients |
| | | taking antibiotics |
| | Use ratio of antiviral drugs | Use ratio of antiviral drugs |
| | Use ratio of a combination of antibacterial | Use ratio of a combination of antibacterial drugs with antiviral |
| | drugs with antiviral drugs | drugs |
| | Use ratio of acetaminophen or ibuprofen | Use ratio of acetaminophen or ibuprofen among child patients |
| | among child patients taking antipyretics | taking antipyretics |
| | Use ratio of a combination of systemic | Use ratio of a combination of systemic glucocorticoids with |
| | glucocorticoids with β-receptor agonists | β-receptor agonists |
| Usage and | | Use intensity of antibacterial drugs |
| dosage | | Use intensity of β-lactam antibiotics |
| | | Use intensity of macrolide antibiotics |
| | | Use intensity of third-generation cephalosporin antibiotics |
| | Use ratio of intravenous administration of | Use ratio of intravenous administration of antibiotics |
| | antibiotics | |
| | Use ratio of sequential therapy | Use ratio of sequential therapy |
| | Use ratio of intravenous administration of | Use ratio of intravenous administration of antiviral drugs |
| | antiviral drugs | |
| | Use ratio of injection among child patients | Use ratio of injection among child patients taking Chinese patent |
| | taking Chinese patent drugs | drugs |
| | Proportion of inhaled corticosteroids (ICS) in | Proportion of ICS in administered glucocorticoids |
| | administered glucocorticoids | |
| Drug use | | Average number of days for antibiotic treatment |
| course | | For child patients with specific pathogenic bacteria, average time |
| | | of extended antibiotic treatment |
| | | Average time of antibiotic use in child patients with |
| | | parapneumonic effusion (PPE) or empyema |
| | | Average number of days for antiviral drug use |
| | | Average time of systemic glucocorticoid use |

RDU assessment indicators for PPIs in children are shown in Table 3 below:

TABLE 3

| | Pediatric intensive care unit (PICU) |
|---|---|
| Drug | Use ratio of PPIs among child patients in PICU |
| selection | Use ratio of PPIs among child patients under |
| | one year old in PICU |
| | Use ratio of PPIs among child patients taking |
| | glucocorticoids in PICU |
| | Use ratio of PPIs among child patients |
| | taking non-steroidal anti-inflammatory drugs (NSAIDs) |
| | Use ratio of PPIs among child patients with |
| | gastro-esophageal reflux diseases (GERDs) in PICU |
| | Use ratio of PPIs among child patients w ith sepsis |
| | Use ratio of PPIs among child patients using ventilators |
| Usage and | Use intensity of PPIs in PICU |
| dosage | Proportion of cumulative DDDs of omeprazole in |
| | cumulative DDDs of PPI in child patients in PICU |
| | during the same period |
| Drug use | Average number of days for PPI use in child |
| course | patients in PICU |

Calculations of the RDU assessment indicators for children with PNS are shown in Table 4 below:

TABLE 4

| | | |
|---|---|---|
| Drug | Use ratio of antibacterial drugs | Number of child patients taking antibacterial drugs/total |
| selection | | number of child patients |
| | Use ratio of RAS inhibitors among | Number of child patients taking RAS inhibitors/number of |
| | non-hypertensive child patients | non-hypertensive child patients |
| | Use ratio of albumin | Number of child patients taking albumin/total number of child |
| | | patients |
| | Use ratio of immunopotentiators | Number of child patients taking immunopotentiators/total |
| | | number of child patients |
| | Use ratio of CNIs | Number of child patients taking CNIs/total number of child |
| | | patients |
| | Use ratio of calcium supplement treatment | Number of child patients undergoing calcium supplement |
| | | treatment/total number of child patients |
| | Use ratio of preferred prednisone (prednisolone) | Number of child patients undergoing preferred prednisone |
| | among child patients undergoing hormone | (prednisolone) treatment/number of child patients undergoing |
| | treatment | hormone treatment |
| | Use ratio of pulse hormone therapy | Number of child patients undergoing pulse hormone |
| | | therapy/total number of child patients |
| | Use ratio of CP among child patients with | Number of child patients undergoing CP treatment/number of |
| | hormone-sensitive and frequent-relapse PNS that | child patients with hormone-sensitive and frequent-relapse PNS |
| | are administered with immunosuppressants | that are administered with immunosuppressants |
| | Use ratio of anticoagulation preventive measures | Number of child patients undergoing anticoagulation preventive |
| | (defined measures: warfarin, dipyridamole, | measures/total number of child patients |
| | Clopidogrel, aspirin, and LMWH) | |
| Usage and | Initial hormone treatment: >60 mg/d | Number of child patients undergoing initial hormone |
| dosage | | treatment >60 mg/d/number of child patients taking hormones |
| | Use ratio of intravenous administration among | Number of child patients intravenously administered with |
| | child patients taking diuretics | diuretics/number of child patients administered with diuretics |
| | Use ratio of CP intravenous injection among child | Number of child patients intravenously injected with |
| | patients taking CP | CP/number of child patients taking CP |
| | Use ratio of a combination of an ACEI/ARB with | Number of child patients administered with a combination of an |
| | a diuretic | ACEI/ARB with a diuretic/total number of child patients |
| | During initial treatment (three days before | Number of child patients initially treated with diuretics/total |
| | admission), use ratio of diuretics | number of child patients |
| | Use ratio of blood concentration monitoring | Number of child patients taking CNI drugs that undergo blood |
| | among patients taking CNI drugs | concentration monitoring/number of child patients taking CNI |
| | | drugs |

Calculations of RDU assessment indicators for children with CAP are shown in Table 5 below:

TABLE 5

| | | |
|---|---|---|
| Drug | Use ratio of antibiotics | Number of child patients taking antibacterial drugs/total number of child |
| selection | | patients |
| | Use ratio of broad-spectrum antibiotics | Number of child patients taking broad-spectrum antibiotics after pathogenic |
| | after pathogenic bacteria are identified | bacteria are identified/number of child patients whose pathogenic bacteria |
| | | are identified |
| | Use ratio of preferred β-lactam | Number of child patients taking preferred β-lactam antibiotics/number of |
| | antibiotics | child patients taking antibiotics |
| | Use ratio of macrolide antibiotics | Number of hospitalized child patients taking macrolide antibiotics/number |
| | | of hospitalized child patients |
| | Use ratio of combined antibiotics | Number of child patients taking combined antibiotics/number of child |
| | | patients taking antibiotics |
| | Proportion of a combination of | Number of child patients taking a combination of macrolides and |
| | macrolides and β-lactams in drug | β-lactams/number of child patients taking a drug combination |
| | combination | |
| | Use ratio of third-generation | Number of hospitalized child patients taking third-generation cephalosporin |
| | cephalosporin antibiotics | antibiotics/number of hospitalized child patients taking antibiotics |
| | Use ratio of vancomycin or | Number of hospitalized child patients taking vancomycin or clindamycin |
| | clindamycin when pathogenic bacteria | when pathogenic bacteria are identified as S. aureus/number of hospitalized |
| | are identified as S. aureus | child patients whose pathogenic bacteria are identified as S. aureus |
| | Microbiological examination rate | Number of child patients taking antibacterial drugs that undergo |
| | among CAP child patients taking | microbiological examination/number of child patients taking antibacterial |
| | antibiotics | drugs |
| | Use ratio of antiviral drugs | Number of child patients taking antiviral drugs/total number of child |
| | | patients |
| | Use ratio of a combination of | Number of child patients taking a combination of antibacterial drugs with |
| | antibacterial drugs with antiviral drugs | antiviral drugs/number of child patients taking antibacterial drugs |
| | Use ratio of acetaminophen or | Number of child patients taking acetaminophen or ibuprofen as an |
| | ibuprofen among child patients taking | antipyretic/number of child patients taking antipyretics |
| | antipyretics | |
| | Use ratio of a combination of systemic | Number of hospitalized child patients taking a combination of systemic |
| | glucocorticoids with β-receptor | glucocorticoids with β-receptor agonists/number of child patients taking |
| | agonists | systemic glucocorticoids |
| Usage and | Use frequency of antibacterial drugs | Σ(use amount of various antibacterial drugs/DDD) |
| dosage | Use frequency of β-lactam antibiotics | Use amount of β-lactam antibiotics/DDD |
| | Use frequency of macrolide antibiotics | Use amount of macrolide antibiotics/DDD |
| | Use frequency of third-generation | Use amount of third-generation cephalosporin antibiotics/DDD |
| | cephalosporin antibiotics | |
| | Use ratio of intravenous administration | Number of child patients intravenously administered with antibacterial |
| | of antibiotics | drugs/number of child patients taking antibacterial drugs |
| | Use ratio of sequential therapy | Number of child patients undergoing sequential therapy/number of child |
| | | patients taking antibiotics |
| | Use ratio of intravenous administration | Number of child patients intravenously administered with antiviral |
| | of antiviral drugs | drugs/number of child patients taking antiviral drugs |
| | Use ratio of injection among child | Number of child patients taking Chinese patent drug injection/number of |
| | patients taking Chinese patent drugs | child patients taking Chinese patent drugs |
| | Proportion of ICS in administered | Number of child patients taking ICS/number of child patients taking |
| | glucocorticoids | glucocorticoids |
| Drug use | Average number of days for antibiotic | Total time of antibacterial drug treatment/total number of child patients |
| course | treatment | receiving antibacterial drug treatment |
| | For child patients with specific | Total time of extended antibiotic treatment for child patients with specific |
| | pathogenic bacteria, average time of | pathogenic bacteria/total number of child patients with specific pathogenic |
| | extended antibiotic treatment | bacteria that undergo extended antibiotic treatment |
| | Average time of antibiotic use in child | Total time of antibiotic use in child patients with PPE or empyema/total |
| | patients with PPE or empyema | number of child patients with PPE or empyema taking antibiotics |
| | Average number of days for antiviral | Total time of antiviral drug treatment/total number of child patients |
| | drug use | receiving antiviral drug treatment |
| | Average time of systemic | Total time of systemic glucocorticoid use/number of child patients taking |
| | glucocorticoid use | systemic glucocorticoids |

Calculations of RDU assessment indicators for PPIs in children are shown in Table 6 below:

TABLE 6

| | | |
|---|---|---|
| Drug | Use ratio of PPIs among child patients in PICU | Number of child patients initially treated with |
| selection | | diuretics/total number of child patients in PICU |
| | Use ratio of PPIs among child patients under one year | Number of child patients under one year old in PICU |
| | old in PICU | that take PPIs/total number of child patients under one |
| | | year old in PICU |
| | Use ratio of PPIs among child patients taking | Number of child patients taking both glucocorticoids |
| | glucocorticoids in PICU | and PPIs in PICU/total number of child patients taking |
| | | glucocorticoids in PICU |
| | Use ratio of PPIs among child patients | Number of child patients taking both NSAIDs and |
| | taking non-steroidal anti-inflammatory drugs (NSAIDs) | PPIs/total number of child patients taking NSAIDs in |
| | | PICU |
| | Use ratio of PPIs among child patients with GERDs in | Number of child patients with GERDs taking PPIs in |
| | PICU | PICU/total number of child patients with GERDs in |
| | | PICU |
| | Use ratio of PPIs among child patients with sepsis | Number of child patients with sepsis taking PPIs/total |
| | | number of child patients with sepsis in PICU |
| | Use ratio of PPIs among child patients using ventilators | Number of child patients taking both NSAIDs and |
| | | PPIs/total number of child patients taking NSAIDs in |
| | | PICU |
| Usage and | Use intensity of PPIs in PICU | Use amount of PPIs/DDD |
| dosage | Proportion of cumulative DDDs of omeprazole in | (use amount of omeprazole/DDD)/Σ(various |
| | cumulative DDDs of PPI in child patients in PICU | PPIs/DDD) |
| | during the same period | |
| Drug use | Average number of days for PPI use in child patients in | Total time of PPI use/total number of child patients in |
| course | PICU | PICU |

### EXAMPLE 3

This example provides a system for assessing drug use in children, as shown in FIG. 2, including:

a building unit configured to build an RDU assessment model for clinical drug use in children;

an acquisition unit configured to acquire clinical drug use data of children and extract classification information therefrom;

a comparison unit configured to import the clinical drug use data of children correspondingly into the RDU assessment model for data comparison according to the classification information to obtain comparison results; and

a summarization unit configured to summarize all comparison results according to the classification information to generate rationality assessment results for clinical drug use in children.

In a possible design, the acquisition unit may be also configured to acquire existing indicator data for drug use in children; and the building unit may be also configured to build an RDU assessment model for clinical drug use in children, and specifically configured to:

extract key information from the existing indicator data for drug use in children, where, the key information may include child age group information, disease burden information, drug metabolism information, and drug use characteristic information; and

establish an assessment indicator system for RDU in children according to the key information, and build the RDU assessment model based on the assessment indicator system for RDU in children.

In a possible design, the system may further include:

an acquisition unit configured to use the Delphi method to obtain corrected data; and

a correction unit configured to use the corrected data to correct and optimize the assessment indicator system for RDU in children.

In a possible design, the system may further include:

a determination unit configured to determine rationality assessment results for clinical drug use in children in various hospitals;

a generation unit configured to generate corresponding rationality assessment reports for clinical drug use in children according to the rationality assessment results for clinical drug use in children in various hospitals; and

a feedback unit configured to feed the corresponding rationality assessment reports for clinical drug use in children back to the hospitals, respectively.

### EXAMPLE 4

This example provides a computing device, as shown in FIG. 3, including:

a memory configured to store instructions; and

a processor configured to read the instructions stored in the memory and execute the method for assessing drug use in children of Example 1 based on the instructions.

The processor may include, but is not limited to, a microprocessor of the STM32F105 series; and the memory may include, but is not limited to, random access memory (RAM), read only memory (ROM), flash memory, First Input First Output (FIFO), and/or First In Last Out (FILO).

### EXAMPLE 5

This example provides a computer-readable storage medium. Instructions are stored on the computer-readable storage medium, and when the instructions are run on a computer, the computer executes the method for assessing drug use in children of Example 1. The computer-readable storage medium refers to a carrier that stores data, which may include, but is not limited to, a floppy disk, an optical disk, a hard disk, a flash memory, a USB flash drive, and/or a memory stick.

The computer may be a general-purpose computer, a dedicated computer, a computer network, or another programmable device.

### EXAMPLE 6

This example provides a computer program product with instructions. When the instructions are run on a computer, the computer executes the method for assessing drug use in children of Example 1. The computer may be a general-purpose computer, a dedicated computer, a computer network, or another programmable device.

Through the above description of the examples, those skilled in the art can clearly understand that the examples can be implemented by means of software in combination with a necessary universal hardware platform, or certainly, can be implemented through hardware. Based on such understanding, the technical solution, in essence, or the part contributing to the prior art may be embodied as a software product. The computer software product may be stored in a computer-readable storage medium, such as ROM/RAM, magnetic disk, or optical disk, and includes a plurality of instructions for making a computing device to implement the method in the examples or parts of the examples.

The present disclosure is not limited to the above-mentioned optional implementations, and anyone can derive other products in various forms under the enlightenment of the present disclosure. The above-mentioned specific examples should not be construed as limiting the protection scope of the present disclosure, and the protection scope of the present disclosure should be defined by the claims. Moreover, the description can be used to interpret the claims.

What is claimed is:

1. A method for assessing drug use in children, comprising:
building a rational drug use (RDU) assessment model for clinical drug use in children;
acquiring clinical drug use data of children and extracting classification information therefrom;
according to the classification information, importing the clinical drug use data of children correspondingly into the RDU assessment model for data comparison to obtain comparison results; and
summarizing all comparison results according to the classification information to generate rationality assessment results for clinical drug use in children.

2. The method for assessing drug use in children according to claim 1, wherein, the building an RDU assessment model for clinical drug use in children comprises:
acquiring existing indicator data for drug use in children;
extracting key information from the existing indicator data for drug use in children, wherein, the key information comprises child age group information, disease burden information, drug metabolism information, and drug use characteristic information; and
establishing an assessment indicator system for RDU in children according to the key information, and building the RDU assessment model based on the assessment indicator system for RDU in children.

3. The method for assessing drug use in children according to claim 2, wherein, the method further comprises:
using the Delphi method to obtain corrected data; and
using the corrected data to correct and optimize the assessment indicator system for RDU in children.

4. The method for assessing drug use in children according to claim 1, wherein, the acquiring clinical drug use data of children comprises:
acquiring a prescription for clinical drug use in children;
reading a content in the prescription for drug use in children to obtain basic information about clinical drug use in children; and
sorting the basic information about clinical drug use in children to obtain the clinical drug use data of children.

5. The method for assessing drug use in children according to claim 1, wherein, the method further comprises:
determining rationality assessment results for clinical drug use in children in various hospitals;
according to the rationality assessment results for clinical drug use in children in various hospitals, generating corresponding rationality assessment reports for clinical drug use in children; and
feeding the corresponding rationality assessment reports for clinical drug use in children back to the hospitals, respectively.

6. The method for assessing drug use in children according to claim 5, wherein, the classification information extracted from the clinical drug use data of children comprises drug selection information, drug use course information, usage and dosage information, and drug cost information.

7. A system for assessing drug use in children, comprising:
a building unit configured to build an RDU assessment model for clinical drug use in children;
an acquisition unit configured to acquire clinical drug use data of children and extract classification information therefrom;
a comparison unit configured to import the clinical drug use data of children correspondingly into the RDU assessment model for data comparison according to the classification information to obtain comparison results; and
a summarization unit configured to summarize all comparison results according to the classification information to generate rationality assessment results for clinical drug use in children.

8. The system for assessing drug use in children according to claim 7, wherein, the acquisition unit is also configured to acquire existing indicator data for drug use in children; and the building unit is also configured to build an RDU assessment model for clinical drug use in children, and specifically configured to:
extract key information from the existing indicator data for drug use in children, wherein, the key information comprises child age group information, disease burden information, drug metabolism information, and drug use characteristic information; and
establish an assessment indicator system for RDU in children according to the key information, and build the RDU assessment model based on the assessment indicator system for RDU in children.

9. A computing device, comprising:
a memory configured to store instructions; and
a processor configured to read the instructions stored in the memory and execute the method according to claim 1 based on the instructions.

10. The computing device according to claim 9, wherein, the building an RDU assessment model for clinical drug use in children comprises:
acquiring existing indicator data for drug use in children;
extracting key information from the existing indicator data for drug use in children, wherein, the key information comprises child age group information, disease burden information, drug metabolism information, and drug use characteristic information; and
establishing an assessment indicator system for RDU in children according to the key information, and building the RDU assessment model based on the assessment indicator system for RDU in children.

11. The computing device according to claim 10, wherein, the method further comprises:
using the Delphi method to obtain corrected data; and
using the corrected data to correct and optimize the assessment indicator system for RDU in children.

12. The computing device according to claim 9, wherein, the acquiring clinical drug use data of children comprises:
acquiring a prescription for clinical drug use in children;
reading a content in the prescription for drug use in children to obtain basic information about clinical drug use in children; and
sorting the basic information about clinical drug use in children to obtain the clinical drug use data of children.

13. The computing device according to claim 9, wherein, the method further comprises:
determining rationality assessment results for clinical drug use in children in various hospitals;
according to the rationality assessment results for clinical drug use in children in various hospitals, generating corresponding rationality assessment reports for clinical drug use in children; and
feeding the corresponding rationality assessment reports for clinical drug use in children back to the hospitals, respectively.

14. The computing device according to claim 13, wherein, the classification information extracted from the clinical drug use data of children comprises drug selection information, drug use course information, usage and dosage information, and drug cost information.
